(19) United States
(12) Patent Application Publication
(10) Pub. No.: US 2019/0233505 A1
(43) Pub. Date: Aug. 1, 2019

(54) METHODS OF TREATING OR PREVENTING ZIKA VIRUS INFECTION

(71) Applicant: Dana-Farber Cancer Institute, Inc., Boston, MA (US)

(72) Inventor: Wayne A. Marasco, Wellesley, MA (US)

(21) Appl. No.: 16/330,942

(22) PCT Filed: Sep. 6, 2017
(86) PCT No.: PCT/US2017/050327

§ 371 Date: Mar. 6, 2019

Related U.S. Application Data
(60) Provisional application No. 62/383,668, filed on Sep. 6, 2016.

(51) Int. Cl. C07K 16/10; A61K 39/395; A61P 31/14

(52) U.S. Cl. CPC C07K 16/1081; A61P 31/14; A61K 39/395

(57) ABSTRACT

The present invention provides antibodies that neutralize flavivirus and methods of use thereof. These antibodies are derived from mAb-11 which recognizes West Nile virus E protein and is cross-reactive with members of the flavivirus family, including dengue virus. The antibodies of the present invention prevent antibody-dependent enhancement of a viral infection by having a modified Fc region that does not bind to the Fey receptor. The invented antibody is used to treat flaviviral infections and symptoms thereof.

### RELATED APPLICATIONS

This application claims benefit of, and priority to, U.S. Ser. No. 62/383,668 filed on Sep. 6, 2016 the contents of which are hereby incorporated by reference its entirety.

### GOVERNMENT INTEREST

This invention was made with government support under AI0703431 awarded by the National Institutes of Health. The United States government has certain rights in the invention.

### FIELD OF THE INVENTION

This invention is directed to methods for the treatment and prevention of viral infections, particularly Zika virus.

### BACKGROUND OF THE INVENTION

Flavivirus is a genus of the family Flaviviridae. The flavivirus genus incorporates over 60 closely related viruses including several human pathogens of global and local epidemiological importance such as dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, Zika virus and Hepatitis C virus.

Flaviviruses, such as Zika virus, present a significant threat to global health. The infection known as Zika fever or Zika virus disease often causes no or only mild symptoms, similar to a very mild form of dengue fever. While there is no specific treatment, acetaminophen and rest may help with the symptoms. As of 2016, the illness cannot be prevented by medications or vaccines. Further, in adults Zika virus has been linked to neurological diseases such as Guillain-Barre Syndrome, encephalitis and others. In infections during pregnancy, Zika virus has been determined to cause a spectrum of birth defects such as microcephaly, fetal brain disruption sequence, brain lesions and calcification, damage to the visual and aural systems, skeletal and muscular damage, seizures, and a range of developmental defects.

Accordingly, there is an urgent need for therapeutics and methods for preventing and treating Zika virus infection.

### SUMMARY OF THE INVENTION

The invention is based upon the discovery of monoclonal antibodies which bind and neutralize flavivirus, and do not contribute to the antibody-dependent enhancement of flavivirus infection.

In one aspect the invention provides a method of treating or preventing of a Zika virus infection by administering to a subject in need thereof a composition containing a fully human monoclonal antibody having a heavy chain with three CDRs comprising the amino acid sequences GYSTH (SEQ ID NO:21), WDNPSSGDTTYAENFRG (SEQ ID NO:22), and GGDDYSFDH (SEQ ID NO:23) respectively; a light chain with three CDRs comprising the amino acid sequences RGDSLRSYYAS (SEQ ID NO:24), GENNRPS (SEQ ID NO:25), and NSRDSSDHLLL (SEQ ID NO:25) respectively; and a modified Fc region such that the Fc region does not bind to the Fcγ receptor. In some embodiments, the fully human monoclonal antibody has a V_{H} amino acid sequence having SEQ ID NO: 1, a V_{L} amino acid sequence having SEQ ID NO: 3 and a modified Fc region such that the Fc region does not bind to the Fcγ receptor. In other embodiments, the fully human monoclonal antibody comprises a V_{H} nucleotide sequence having SEQ ID NO: 2, a V_{L} nucleotide sequence having SEQ ID NO: 4, and a modified Fc region such that the Fc region does not bind to the Fcγ receptor. Preferably, the fully human monoclonal antibody has a heavy chain amino acid sequence having SEQ ID NO: 20 and a V_{L} amino acid sequence having SEQ ID NO: 3.

The fully human monoclonal antibody has an Fc region containing mutations at amino acid positions 234 and 235. For example, the mutations are L234A and L235A. In some aspects the fully human monoclonal antibody has a Fc region amino acid sequence of SEQ ID NO: 7 or SEQ ID NO: 13. In other aspects, the modified Fc region binds to the neonatal Fc receptor.

Other features and advantages of the invention will be apparent from and are encompassed by the following detailed description and claims.

### BRIEF DESCRIPTION OF THE DRAWINGS

FIG. 1 shows neutralization of Zika Virus strain MR766, Uganda/1947/Africa genotype by AV-1 (mAb-11 LALA) using the PRNT Assay (BHK-21 cells)

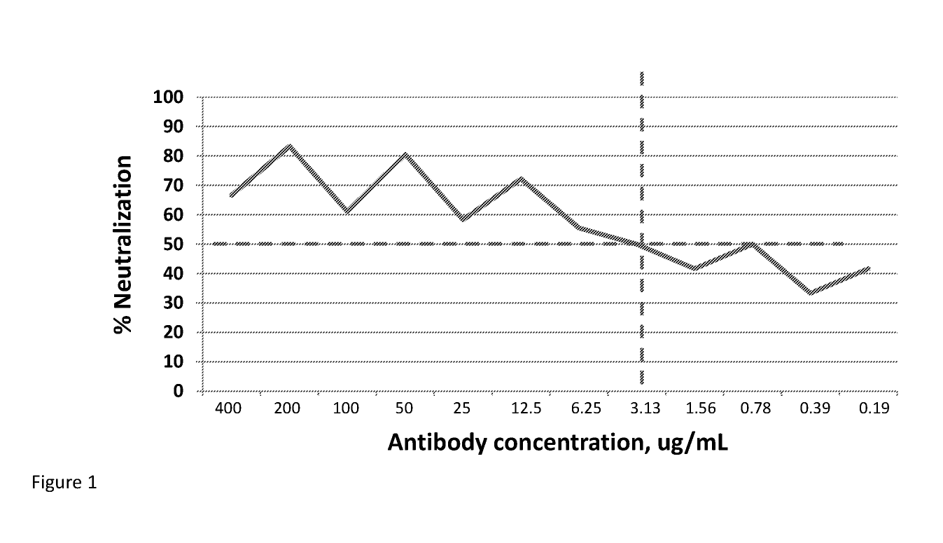

FIG. 2 shows neutralization of Zika Virus strain FSS13025, Cambodia/2010/Asian genotype by AV-1 (mAb-11 LALA) using the FACS neutralization Assay (U937-DC-SIGN cells).

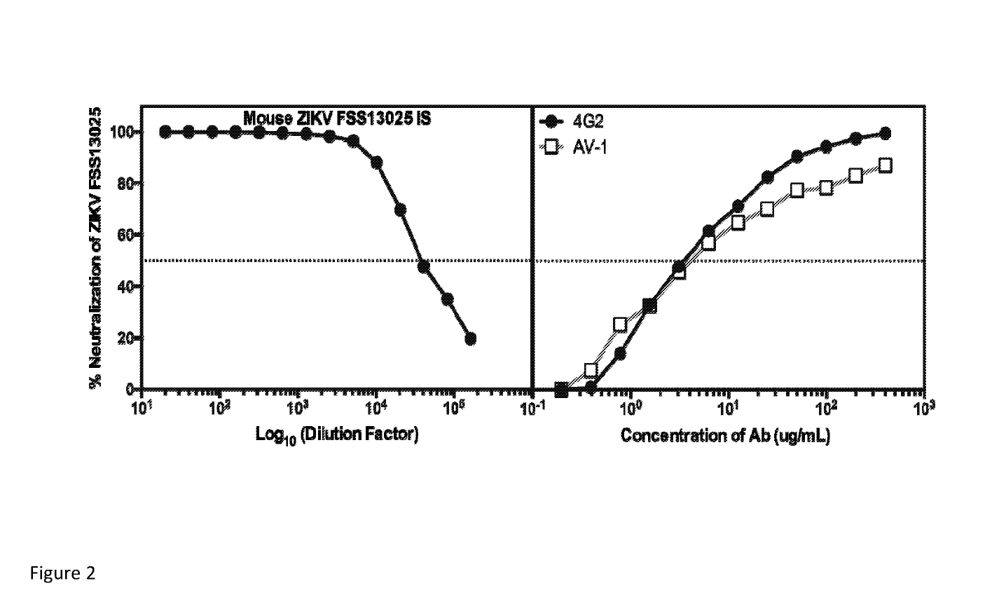

FIG. 3 illustrates the AV-1 (mAb-11 LALA) recognizes a common domain in the Flavivirus E-protein.

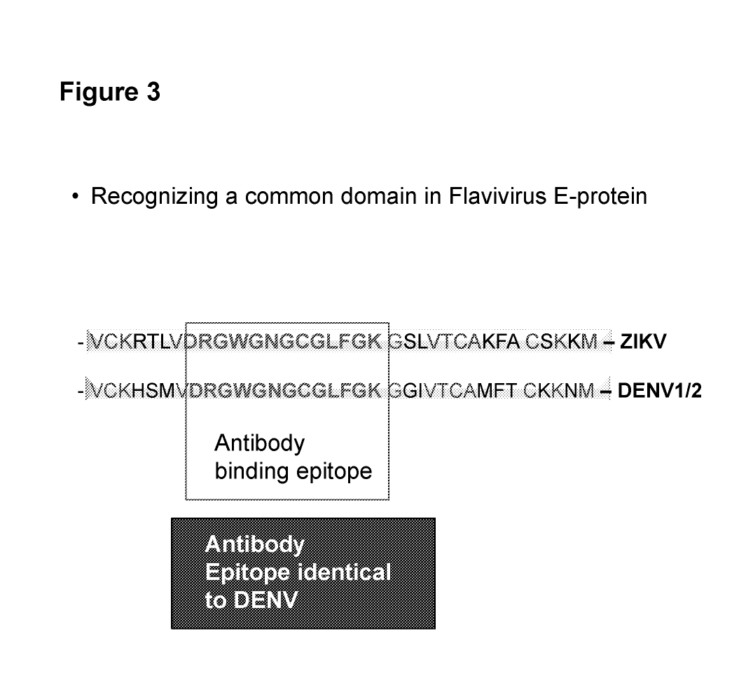

FIG. 4 shows the neutralization capacity of AV-1 (mAb-11 LALA) AV-1 has in vitro activity against multiple flaviviruses.

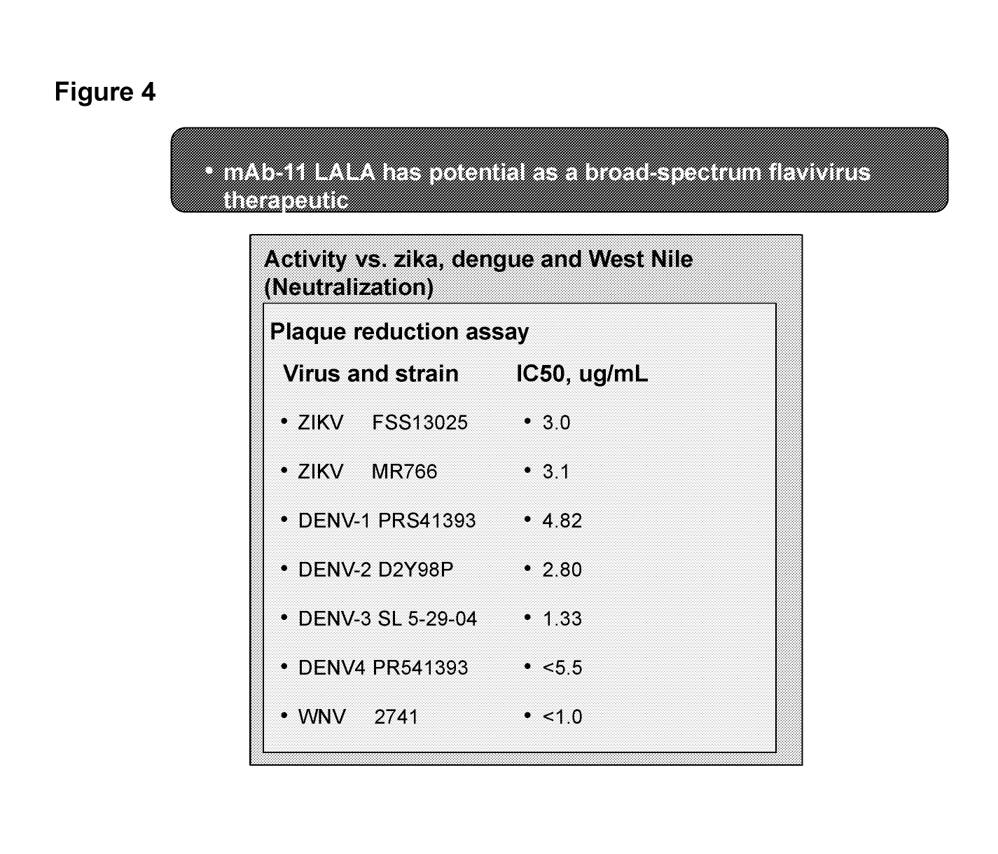

### DETAILED DESCRIPTION

The present invention provides antibodies that neutralize Zika virus infection without contributing to antibody-dependent enhancement of virus infection. Antibodies that bind and neutralize Zika virus are described in International Publication WO 2005/123774, the contents of which are incorporated by reference in its entirety. The antibodies of the present invention were produced by modifying an antibody against West Nile virus, mAb-11, such that the Fc region of the antibody does not bind to the Fc-gamma receptor and complement component C1q. Thus, the modified antibody does not contribute to antibody-dependent enhancement of infection. The antibodies and methods disclosed herein relate to this antibody, and methods for treating or preventing a Zika virus infection. The antibody of the present invention, mAb-11 LALA, has demonstrated increased capability of preventing and treating Zika virus infection compared to the wild-type mAb-11, as described herein and demonstrated in the examples.

The invention provides for both prophylactic and therapeutic methods of treating a subject at risk of (or susceptible to) Zika virus infection or a Zika-virus related disease or disorder.

In one aspect, the invention provides methods for preventing a Zika virus infection or a Zika-virus related disease or disorder in a subject by administering to the subject a monoclonal antibody of the invention. For example, monoclonal antibody mAb-11, mAb-11 LALA, and any variants thereof, wherein the Fc region is modified thereby reducing or abrogating binding to the Fc-gamma receptor and also to complement (C1q), may be administered in therapeutically effective amounts. Optionally, two or more anti-flavivirus antibodies are co-administered.

The subject is e.g., any mammal, e.g., a human, a primate, mouse, rat, dog, cat, cow, horse, or pig.

Subjects at risk for Zika virus-related diseases or disorders include patients who have been exposed to the flavivirus from an infected arthropod (i.e., mosquito or tick). Subjects at risk for Zika virus-related diseases or disorders also include those who have been exposed to carriers of Zika virus through sex and/or other routes of exposure. For example, the subjects have traveled to regions or countries of the world in which Zika and/or other flavivirus infections have been reported and confirmed. Administration of a prophylactic agent can occur prior to the manifestation of symptoms characteristic of the Zika virus-related disease or disorder, such that a disease or disorder is prevented or, alternatively, delayed in its progression.

Another aspect of the invention pertains to methods of treating a Zika virus infection in a subject. Zika virus infections are treated by administering to the subject a monoclonal antibody of the invention. For example, monoclonal antibody mAb-11, mAb11-LALA, and any variants thereof, wherein the Fc region is modified thereby reducing or abrogating binding to the Fc-gamma receptor and/or complement (e.g. C1q), may be administered in therapeutically effective amounts. Optionally, two or more anti-flavivirus antibodies are co-administered.

The invention provides for treating a flavivirus-related disease or disorder, such as West Nile fever, meningitis, dengue fever, yellow fever or encephalitis, in a patient by administering two or more antibodies, such as mAb-11 LALA or a variant of mAb-11, wherein the Fc region of said variant does not bind or has reduced binding to the Fc-gamma receptor, with other flavivirus neutralizing antibodies known in the art, such as mAb-11. In another embodiment, the invention provides methods for treating a flavivirus-related disease or disorder in a patient by administering an antibody of the present invention, such as mAb-11 LALA or a mAb-11 variant as described herein, with any anti-viral agent known in the art. Anti-viral agents can be peptides, nucleic acids, small molecules, inhibitors, or RNAi.

The methods described herein lead to a reduction in the severity or the alleviation of one or more symptoms of a viral infection. Infections are diagnosed and/or monitored, typically by a physician using standard methodologies.

The treatment is administered prior to diagnosis of the infection. Alternatively, treatment is administered after diagnosis. Efficaciousness of treatment is determined in association with any known method for diagnosing or treating the particular disorder or infection. Alleviation of one or more symptoms of the disorder indicates that the compound confers a clinical benefit.

Neutralizing antibodies have been and are being currently developed for the treatment and prevention of viral infections, specifically infections by members of the Flavivirus genus. Initial studies have demonstrated that such antibodies show increased neutralization and protection from infection by flavivirus family members (i.e., West Nile Virus or one of the four dengue virus serotypes). However, subsequent virus challenge studies, in which experimental subjects were treated with neutralizing antibodies and then challenged with doses of flavivirus (i.e., dengue virus), did not show a decrease in viremia. In some cases, treatment with such antibodies resulted in enhancement of infection compared to controls, which is believed to be mediated though a mechanism called antibody-dependent enhancement (ADE). These results demonstrate the importance of developing therapeutics and methods that prevent antibody-dependent enhancement of flavivirus infection.

Antibody-dependent enhancement of infection can be accomplished by the binding of the Fc region of the antibody to an Fcγ receptor (FcγR) on a host cell. Infectious viral particles bound to these antibodies are therefore more efficiently brought to host cells by Fc region-Fc receptor binding. This increases the infection and replication rate of the virus, thereby enhancing the infectivity and pathogenicity of the virus. ADE can also be accomplished by the binding of the Fc region of the antibody to complement.

In contrast to standard anti-viral antibodies, the antibodies of the present invention have reduced binding to the Fcγ receptors (FcγR) or do not bind to the FcγR. Fcγ receptors include, for example, FcγRI, FcγRIIIa, FcγRIIIb, and FcγRIIIc. Further, the antibodies of the present invention have reduced binding or do not bind to complement (e.g. components such as C1q). In one embodiment, the antibodies of the invention contain one or more mutations in the Fc region. The mutation(s) may be any mutation that reduces or abrogates binding of the antibody to an FcγR and/or to complement. Mutations can be substitutions, additions, or deletions of amino acids in the Fc region. Although the antibodies of the present invention have mutated Fc regions, the antibodies still confer potent flavivirus neutralization.

The Fc region of an antibody comprises two domains, CH2 and CH3. These domains, or specific amino acids within these domains known in the art, mediate the interaction with FcγR. Antibodies of the present invention contain any mutation (i.e., substitution, addition, or deletion of one or more than one amino acid) in the CH2 or CH3 domain, or both, that reduces or abrogates the binding of the antibody to an FcγR. For example, antibodies of the present invention contain a mutation or substitution of at least one amino acid at positions 233, 234, 235, 236, 237, 250, 314, or 428 of the wild-type Fc region. Preferably, the amino acid substitution is to an alanine.

In one embodiment, the Fc region of an antibody of the invention comprises a substitution at positions 234 or 235 of the heavy chain of the antibody, or both. In general, the amino acid at positions 234 and 235 of the wild-type Fc region is a leucine (“L”). In one embodiment, the antibodies of the invention comprise an amino acid at position 234, 235, or both, that is not a leucine. In another embodiment, the antibodies of the invention comprise an alanine (“A”) at position 234, 235 or both. An antibody comprising the mutations at positions 234 and 235 of the Fc region where the leucines are mutated to alanines is referred to herein as a “LALA” variant.

In a preferred embodiment, the antibodies of the present invention are full length, or intact, antibodies, wherein the antibodies contain an antigen-binding region (i.e., Fab region or Fab fragment) and an Fc region (modified or mutated, as described herein). Previously developed antibodies in the art that were designed to circumvent ADE often lack the Fc region to prevent binding to FcγR. Antibodies of the present invention provide superior properties by retaining the Fc region. One such property is the ability to bind to the neonatal receptor (FcRn) expressed on endothelial cells, which plays a critical role in the homeostasis of circulating IgG levels. Binding of circulating antibodies to the FcRn induces internalization through pinocytosis, in which the antibodies are recycled to the cell surface, and released at the basic pH of blood. This mechanism protects the antibodies of the present invention from degradation and increases the half-life compared to other unmodified antibodies or antibody fragments lacking the Fc region. Increased persistence of the antibodies of the present invention in the serum provides increased efficacy by allowing higher circulating levels, less frequent administration, and reduced doses.

Another property of the antibodies of the present invention may include the ability to prevent binding to complement factors. Binding of complement factors, such as C1q, to the Fc region of the antibody triggers a signaling cascade to activate complement-dependent cytotoxicity (CDC) and antibody-dependent cell-mediated cytoxicity (ADCC). Avoiding binding of the Fc region of the antibody to complement factors abolishes CDC and ADCC [Hezareh et al., Effector Function Activities of a Panel of Mutants of a Broadly Neutralizing Antibody against Human Immunodeficiency Virus Type 1, Journal of Virology Vol. 75, No. 24, 2001] and avoids ADE of flavivirus disease [see DFCI Patent 1701].

It is known in the art that the binding sites on the Fc region of Fcγ receptors is distinct from the binding site of the neonatal Fc receptor (FcRn). Therefore, the antibodies of the present invention have Fc regions modified such that they have reduced binding or cannot bind to the Fcγ receptors, however are still competent for binding to the FcRn receptor. Antibodies of the invention can be modified by introducing random amino acid mutations into particular regions of the CH2 or CH3 domain of the heavy chain in order to alter their binding affinity for FcγR and/or FcRn and/or their serum half-life in comparison to the unmodified antibodies. Examples of such modifications include, but are not limited to, substitutions of at least one amino acid from the heavy chain region selected from the group consisting of amino acid residues 234, 235, 236, 237, 250, 314, and 428. Accordingly, the antibodies of the present invention have greater half-life than unmodified antibodies, which confers increased efficacy in the prevention and treatment of Zika virus infections and subsequent disease.

In one aspect, the antibodies of the present invention have Fc regions modified such that they have reduced binding or cannot bind to the Fcγ receptors, however are still competent for binding to complement factors, such as C1q.

One of ordinary skill in the art could readily prepare the modified antibodies of the present invention. Recombinant DNA techniques for introducing mutations or substitutions in the Fc region of an antibody are known in the art. Characterization of the Fc region for their ability to bind or not bind to Fc receptors (FcγR or FcRn) and complement factors (C1q) can be readily performed by the ordinarily skilled artisan, for example by immunoprecipitation, immunoassay, affinity chromatography, or array techniques.

The fully-human antibodies described herein may be produced in mammalian expression systems, such as Chinese Hamster Ovary (CHO) cell lines, hybridomas and other systems. The fully-human antibodies described herein may also be produced by non-mammalian expression systems, for example, by transgenic plants. For example, the antibodies described herein are produced in transformed tobacco plants (N. benthamiana and N. tabaccum).

The various nucleic acid and amino acid sequences of variable regions of mAb-11 and mAB-11 LALA are provided below:

Heavy Chain Variable (V_{H}) Amino Acid Sequence:
(SEQ ID NO: 1)
QVQLVQSGAEVKKPGASVKVSCKASGYTFSGYSTHWLRQVPGQGLEWIGW
DNPSSGDTTYAENFRGRVTLTRDTSITTDYLEVRGLRSDDTAVYYCARGG
DDYSFDHWGQGTLVTVSS
Heavy Chain Variable (V_{H}) Nucleic Acid Sequence:
(SEQ ID NO: 2)
CAGGTGCAGCTGGTGCAGTCTGGGGCTGAGGTGAAGAAGCCTGGGGCCTC
AGTGAAAGTCTCCTGCAAGGCTTCTGGATACACCTTCAGCGGCTACTCTA
CACACTGGCTGCGACAGGTCCCTGGACAGGGACTTGAGTGGATTGGATGG
GACAACCCTAGTAGTGGTGACACGACCTATGCAGAGAATTTTCGGGGCAG
GGTCACCCTGACCAGGGACACGTCCATCACCACAGATTACTTGGAAGTGA
GGGGTCTAAGATCTGACGACACGGCCGTCTATTATTGTGCCAGAGGCGGA
GATGACTACAGCTTTGACCATTGGGGTCAGGGCACCCTGGTCACCGTCTC
CTCA
Light Chain Variable (V_{L}) Amino Acid Sequence:
(SEQ ID NO: 3)
SSELTQDPAVSVALGQTVRITCRGDSLRSYYASWYQQKPGQAPVLVIYGE
NNRPSGIPDRFSGSSSGDTASLTITGAQAEDEADYYCNSRDSSDHLLLFG
GGTKLTVLG
Light Chain Variable (V_{L}) Nucleic Acid Sequence:
(SEQ ID NO: 4)
TCTTCTGAGCTGACTCAGGACCCAGCTGTGTCTGTGGCCTTGGGACAGAC
AGTCAGGATCACATGCCGAGGAGACAGCCTCAGAAGTTATTATGCAAGCT
GGTACCAACAGAAGCCAGGACAGGCCCCTGTACTTGTCATCTATGGTGAA
AACAACCGACCCTCAGGGATCCCAGACCGATTCTCTGGCTCCAGCTCAGG
AGACACAGCTTCCTTGACCATCACTGGGGCTCAGGCGGAAGATGAGGCTG
ACTATTACTGTAACTCCCGGGACAGCAGTGATCACCTTCTCCTATTCGGT
GGAGGGACCAAGTTGACCGTCCTAGGT

The Fc region comprises three heavy constant domains, CH1, CH2 or CH3 domains. A hinge region joins the CH1 and CH2 regions. Exemplary Fc region sequences for wild-type and modified Fc regions with respect to the invention are provided below.

The amino acid sequence of the Fc Region of wild-type mAb-11 is provided as follows:

(SEQ ID NO: 5)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The nucleic acid sequence of the Fc Region of wild-type mAb-11 is provided as follows:

(SEQ ID NO: 6)
GCTAGCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAAGCAGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGATGAGCTGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCTCCGGGTAAATGA

The amino acid sequence of the modified Fc region of mAb-11 LALA is provided below. For example, the amino acids at positions 108 and 109 are mutated. In the sequence provided below, the leucine amino acids at position 108 and 109 are mutated to alanines (underlined).

(SEQ ID NO: 7)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The nucleic acid sequence of the modified Fc region of mAb-11 LALA is provided below. For example, the amino acids at positions 108 and 109 encoded by the provided nucleic acid sequence are mutated from leucines to alanines (underlined).

(SEQ ID NO: 8)
GCTAGCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAAGCAGAGCCC
AAATCTTGTGACAAAACTCACACATGCCCACCGTGCCCAGCACCTGAAGC
CGCCGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGATGAGCTGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCTCCGGGTAAATGA

The amino acid sequence for the CH1 region is provided below:

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKK

The nucleic acid sequence for the CH1 region is provided below:

(SEQ ID NO: 10)
GCTAGCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAA

The mAb-11 antibody described herein may comprise a mutation specifically in the CH2 region that reduces or inhibits binding to the Fcγ receptor. Preferably, the mutation does not affect binding to FcRn receptor. Preferably, the mAb-11 antibody contains two mutations in the CH2 region, such that two adjacent leucines are mutated to alanines described below. For example, the mutations are located at amino acid positions 4 and 5 of the CH2 region. Preferably, the mutations are to alanines.

The amino acid sequence for the CH2 region of the wild-type mAb-11 antibody is provided below:

(SEQ ID NO: 11)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK

The nucleic acid sequence for the CH2 region of the wild-type mAb-11 antibody is provided below:

(SEQ ID NO: 12)
GCACCTGAACTCCTGGGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA

The amino acid sequence for the CH2 region of the mutant mAb-11 antibody is provided below (the LALA mutation is underlined):

(SEQ ID NO: 13)
APEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK

The nucleic acid sequence for the CH2 region of the mutant mAb-11 antibody is provided below (the LALA mutation is underlined):

(SEQ ID NO: 14)
GCACCTGAAGCCGCCGGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA

The amino acid sequence for the CH3 region of the mAb-11 antibody is provided below:

(SEQ ID NO: 15)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The nucleic acid sequence for the CH3 region of the mAb-11 antibody is provided below:

(SEQ ID NO: 16)
GGGCAGCCCCGAGAACCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCATGAGGCTCTGCACAACCACTACACGCAGAAGAGC
CTCTCCCTGTCTCCGGGTAAATGA

The amino acid sequence for the hinge region is provided below:

(SEQ ID NO: 17)
AEPKSCDKTHTCPPCP

The nucleic acid sequence for the hinge region is provided below:

(SEQ ID NO: 18)
GCAGAGCCCAAATCTTGTGACAAAACTCACACATGCCCACCGTGCCCA

The amino acid sequence of the heavy chain (including both variable and constant regions) of wild-type mAb-11 is provided below:

(SEQ ID NO: 19)
QVQLVQSGAEVKKPGASVKVSCKASGYTFSGYSTHWLRQVPGQGLEWIGW
DNPSSGDTTYAENFRGRVTLTRDTSITTDYLEVRGLRSDDTAVYYCARGG
DDYSFDHWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKAEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

The amino acid sequence of the heavy chain (including both variable and constant regions) of mutant mAb-11 is provided below (LALA mutation is underlined):

(SEQ ID NO: 20)
QVQLVQSGAEVKKPGASVKVSCKASGYTFSGYSTHWLRQVPGQGLEWIGW
DNPSSGDTTYAENFRGRVTLTRDTSITTDYLEVRGLRSDDTAVYYCARGG
DDYSFDHWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKAEPKSCDKTHTCPPCPAPEAAGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

Antibodies

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of the desired antigen and does not react with other polypeptides. Antibodies include, but are not limited to, polyclonal, monoclonal, and chimeric antibodies.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

As used herein, the term “epitope” includes any protein determinant capable of specific binding to an immunoglobulin, an scFv, or a T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to a flavivirus epitope when the equilibrium binding constant (K_{d}) is ≤1 μM, preferably ≤100 nM, more preferably ≤10 nM, and most preferably ≤100 pM to about 1 pM, as measured by assays such as radioligand binding assays or similar assays known to those skilled in the art.

Those skilled in the art will recognize that it is possible to determine, without undue experimentation, if a human monoclonal antibody has the same specificity as a human monoclonal antibody of the invention by ascertaining whether the former prevents the latter from binding to a flavivirus, e.g. Zika virus. If the human monoclonal antibody being tested competes with the human monoclonal antibody of the invention, as shown by a decrease in binding by the human monoclonal antibody of the invention, then it is likely that the two monoclonal antibodies bind to the same, or to a closely related, epitope.

Various procedures known within the art may be used for the production of polyclonal or monoclonal antibodies directed against a protein of the invention, or against derivatives, fragments, analogs, homologs or orthologs thereof (See, for example, Antibodies: A Laboratory Manual, Harlow E, and Lane D, 1988, Cold Spring Harbor Laboratory Press, Cold Spring Harbor, N.Y., incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

The term “monoclonal antibody” or “mAb” or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of the desired antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “humanized antibodies”, “human antibodies”, or “fully human antibodies” herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, fully human (and humanized) antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Preferred transfer methods and vectors include naked DNA, viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells' cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

In a preferred embodiment, the antibodies of the present invention are full-length antibodies, containing an Fc region similar to wild-type Fc regions that bind to Fc receptors.

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in neutralizing or preventing viral infection. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)). In a preferred embodiment, the antibody of the present invention has modifications of the Fc region, such that the Fc region does not bind to the Fc receptors. Preferably, the Fc receptor is Fcγ receptor. Particularly preferred are antibodies with modification of the Fc region such that the Fc region does not bind to Fcγ, but still binds to neonatal Fc receptor.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, saponaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987)). Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages are in general less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

Pharmaceutical Compositions

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Passive Immunization

Passive immunization has proven to be an effective and safe strategy for the prevention and treatment of viral diseases. (See Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), each of which are incorporated herein by reference). Passive immunization using neutralizing human monoclonal antibodies could provide an immediate treatment strategy for emergency prophylaxis and treatment of Zika virus infection and related diseases and disorders while the alternative and more time-consuming development of vaccines and new drugs is underway.

Subunit vaccines potentially offer significant advantages over conventional immunogens. They avoid the safety hazards inherent in production, distribution, and delivery of conventional killed or attenuated whole-pathogen vaccines. Furthermore, they can be rationally designed to include only confirmed protective epitopes, thereby avoiding suppressive T epitopes (see Steward et al., J. Virol. 69:7668 (1995)) or immunodominant B epitopes that subvert the immune system by inducing futile, non-protective responses (e.g. “decoy” epitopes). (See Garrity et al., J. Immunol. 159:279 (1997)).

Moreover, those skilled in the art will recognize that good correlation exists between the antibody neutralizing activity in vitro and the protection in vivo for many different viruses, challenge routes, and animal models. (See Burton, Natl. Rev. Immunol. 2:706-13 (2002); Parren et al., Adv. Immunol. 77:195-262 (2001)). The data presented herein demonstrate that the mAb-11 human monoclonal antibody, mAb-11 LALA, and other mAb variants can be further developed and tested in in vivo animal studies to determine clinical utility as a potent inhibitor for prophylaxis and treatment of Zika virus infection and related diseases and disorders, while simultaneously avoiding ADE of flavivirus infections.

Antigen-Ig Chimeras in Vaccination

It has been over a decade since the first antibodies were used as scaffolds for the efficient presentation of antigenic determinants to the immune systems. (See Zanetti, Nature 355:476-77 (1992); Zaghouani et al., Proc. Natl. Acad. Sci. USA 92:631-35 (1995)). When a peptide is included as an integral part of an IgG molecule (e.g., the 11A or 256 IgG1 monoclonal antibody described herein), the antigenicity and immunogenicity of the peptide epitopes are greatly enhanced as compared to the free peptide. Such enhancement is possibly due to the antigen-IgG chimeras' longer half-life, better presentation and constrained conformation, which mimic their native structures.

Moreover, an added advantage of using an antigen-Ig chimera is that either the variable or the Fc region of the antigen-Ig chimera can be used for targeting professional antigen-presenting cells (APCs). To date, recombinant Igs have been generated in which the complementarity-determining regions (CDRs) of the heavy chain variable gene (V_{H}) are replaced with various antigenic peptides recognized by B or T cells. Such antigen-Ig chimeras have been used to induce both humoral and cellular immune responses. (See Bona et al., Immunol. Today 19:126-33 (1998)).

Chimeras with specific epitopes engrafted into the CDR3 loop have been used to induce humoral responses to either HIV-1 gp120 V3-loop or the first extracellular domain (D1) of human CD4 receptor. (See Lanza et al., Proc. Natl. Acad. Sci. USA 90:11683-87 (1993); Zaghouani et al., Proc. Natl. Acad. Sci. USA 92:631-35 (1995)). The immune sera were able to prevent infection of CD4 SupT1 cells by HIV-1MN (anti-gp120 V3C) or inhibit syncytia formation (anti-CD4-D1). The CDR2 and CDR3 can be replaced with peptide epitopes simultaneously, and the length of peptide inserted can be up to 19 amino acids long.

Alternatively, one group has developed a “troybody” strategy in which peptide antigens are presented in the loops of the Ig constant (C) region and the variable region of the chimera can be used to target IgD on the surface of B-cells or MHC class II molecules on professional APCs including B-cells, dendritic cells (DC) and macrophages. (See Lunde et al., Biochem. Soc. Trans. 30:500-6 (2002)).

An antigen-Ig chimera can also be made by directly fusing the antigen with the Fc portion of an IgG molecule. You et al., Cancer Res. 61:3704-11 (2001) were able to obtain all arms of specific immune response, including very high levels of antibodies to hepatitis B virus core antigen using this method.

DNA Vaccination

DNA vaccines are stable, can provide the antigen an opportunity to be naturally processed, and can induce a longer-lasting response. Although a very attractive immunization strategy, DNA vaccines often have very limited potency to induce immune responses. Poor uptake of injected DNA by professional APCs, such as dendritic cells (DCs), may be the main cause of such limitation. Combined with the antigen-Ig chimera vaccines, a promising new DNA vaccine strategy based on the enhancement of APC antigen presentation has been reported (see Casares, et al., Viral Immunol. 10:129-36 (1997); Gerloni et al., Nat. Biotech. 15:876-81 (1997); Gerloni et al., DNA Cell Biol. 16:611-25 (1997); You et al., Cancer Res. 61:3704-11 (2001)), which takes advantage of the presence of Fc receptors (FcγRs) on the surface of DCs.

It is possible to generate a DNA vaccine encoding an antigen (Ag)-Ig chimera. Upon immunization, Ag-Ig fusion proteins will be expressed and secreted by the cells taking up the DNA molecules. The secreted Ag-Ig fusion proteins, while inducing B-cell responses, can be captured and internalized by interaction of the Fc fragment with FcγRs on the DC surface, which will promote efficient antigen presentation and greatly enhance antigen-specific immune responses. Applying the same principle, DNA encoding antigen-Ig chimeras carrying a functional anti-MHC II specific scFv region gene can also target the immunogens to all three types of APCs. The immune responses could be further boosted with use of the same protein antigens generated in vitro (i.e., “prime and boost”), if necessary. Using this strategy, specific cellular and humoral immune responses against infection of Zika virus can be accomplished through intramuscular (i.m.) injection of a DNA vaccine. (See Casares et al., Viral. Immunol. 10:129-36 (1997)).

Vaccine Compositions

Therapeutic or prophylactic compositions are provided herein, which generally comprise mixtures of one or more monoclonal antibodies or ScFvs and combinations thereof. The prophylactic vaccines can be used to prevent a Zika virus infection and the therapeutic vaccines can be used to treat individuals following a Zika virus infection. Prophylactic uses include the provision of increased antibody titer to a Zika virus in a vaccination subject. In this manner, subjects at high risk of contracting Zika virus (i.e., in subtropical regions where viral-carrying mosquitos thrive; or sexual partners, caregivers, or other close contacts of persons known or at risk of having Zika virus infections) can be provided with passive immunity to a Zika virus.

These vaccine compositions can be administered in conjunction with ancillary immunoregulatory agents. For example, cytokines, lymphokines, and chemokines, including, but not limited to, IL-2, modified IL-2 (Cys125→Ser125), GM-CSF, IL-12, γ-interferon, IP-10, MIP1β, and RANTES.

Methods of Immunization

The vaccines of the present invention have superior immunoprotective and immunotherapeutic properties over other anti-viral vaccines.

The invention provides a method of immunization, e.g., inducing an immune response, of a subject. A subject is immunized by administration to the subject a composition containing a membrane fusion protein of a pathogenic enveloped virus. The fusion protein is coated or embedded in a biologically compatible matrix.

The fusion protein is glycosylated, e.g. contains a carbohydrate moiety. The carbohydrate moiety may be in the form of a monosaccharide, disaccharide(s). oligosaccharide(s), polysaccharide(s), or their derivatives (e.g. sulfo- or phospho-substituted). The carbohydrate is linear or branched. The carbohydrate moiety is N-linked or O-linked to a polypeptide. N-linked glycosylation is to the amide nitrogen of asparagine side chains and O-linked glycosylation is to the hydroxy oxygen of serine and threonine side chains.

The carbohydrate moiety is endogenous to the subject being vaccinated. Alternatively, the carbohydrate moiety is exogenous to the subject being vaccinated. The carbohydrate moiety(s) are such that are not typically expressed on polypeptides of the subject being vaccinated. For example, the carbohydrate moieties are plant-specific carbohydrates. Plant specific carbohydrate moieties include for example N-linked glycan having a core bound α1,3 fucose or a core bound β 1,2 xylose. Alternatively, the carbohydrate moiety(s) are carbohydrate moieties that are expressed on polypeptides or lipids of the subject being vaccinated. For example, many host cells have been genetically engineered to produce human proteins with human-like sugar attachments.

The invention will be further described in the following examples, which do not limit the scope of the invention described in the claims.

### EXAMPLES

### Example 1: Determination of mAb-11 LALA Efficacy in Neutralization of Zika Virus In Vitro

This study was conducted to determine the efficacy of the human mAb-11 LALA in promoting neutralization of Zika virus. Antibody at multiple concentrations were incubated with Zika virus preparations followed by infection of selected Baby Hamster Kidney cells (BHK-21) or human monocyte cells (U937-DC-SIGN). Neutralization was used to determine the 50% inhibitory dose (IC50).

Target Cells:

BHK-21 cells (Baby Hamster Kidney fibroblasts, ATCC CCL-10), and U937-DC-SIGN (Human monocyte U-937 cells transduced with the gene for the human DC SIGN protein, ATCC CRL-3253).

Virus:

Strain: MR766 (1947 Uganda isolate, African genotype), and strain FSS13025 (2010 Cambodian isolate, Asian genotype).

Plaque Reduction Neutralization Test (PRNT).

BHK-21 cells were infected with 30 plaque-forming units (PFU) of ZIKV strain MR766 in the presence of two-fold dilutions of the human IgG1 monoclonal antibody, mAb-11 LALA. Twelve concentrations were tested starting with a concentration of 400 ug/m and ending with a concentration of 0.19 ug/mL and the plaque reduction neutralization test was incubated for 6 days to determine the 50% inhibitory concentration (IC50).

FACS-Based Neutralization Assay:

Antibodies were diluted using two-fold dilutions, beginning at 1:10 for the dengue immunized mouse positive control serum and 400 ug/mL for the mAb-11 LALA (AV-1) and 4G2 (mouse antibody recognizing dengue envelope protein) monoclonal antibodies. 10⁴ focus-forming units (FFU) of ZIKV strain FSS13025 was mixed with each antibody dilution and incubated for 1 h at 37° C., followed by incubation of the virus/antibody combinations with U937-DC-SIGN cells for 24 h at 37° C. The cells were then fixed and stained intracellularly for the virus using pan flavivirus-specific mAb (clone 4G2) and extracellularly for DC-SIGN. The cells were processed by flow cytometry, and the percentage of cells positive for DC-SIGN and infected with ZIKV was determined.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or preventing of a Zika virus infection, comprising administering to a subject in need thereof a composition comprising a fully human monoclonal antibody comprising:
a. a heavy chain with three CDRs comprising the amino acid sequences GYSTH (SEQ ID NO:21), WDNPSSGDTTYAENFRG (SEQ ID NO:22), and GGDDYSFDH (SEQ ID NO:23) respectively;
b. a light chain with three CDRs comprising the amino acid sequences RGDSLRSYYAS (SEQ ID NO:24), GENNRPS (SEQ ID NO:25), and NSRDSSDHLLL (SEQ ID NO:25) respectively; and
c. a modified Fc region such that the Fc region does not bind to the Fcγ receptor.

2. The method of claim 1, wherein the fully human monoclonal antibody comprises a VII amino acid sequence having SEQ ID NO: 1, a V_{L} amino acid sequence having SEQ ID NO: 3, and a modified Fc region such that the Fc region does not bind to the Fcγ receptor.

3. The method of claim 1, wherein the fully human monoclonal antibody comprises a V_{H} nucleotide sequence having SEQ ID NO: 2, a V_{L} nucleotide sequence having SEQ ID NO: 4, and a modified Fc region such that the Fc region does not bind to the Fcγ receptor.

4. The method of claim 1, wherein the fully human monoclonal antibody comprises a heavy chain amino acid sequence having SEQ ID NO: 20 and a V_{L} amino acid sequence having SEQ ID NO: 3.

5. The method of claim 1, wherein the fully human monoclonal antibody comprises a Fc region containing mutations at amino acid positions 234 and 235.

6. The method of claim 5, wherein the mutations are L234A and L235A.

7. The method of claim 1, wherein the fully human monoclonal antibody comprises a Fc region amino acid sequence of SEQ ID NO: 7 or SEQ ID NO: 13.

8. The method of any of the preceding claims, wherein the modified Fc region binds to the neonatal Fc receptor.
